(19) United States
(12) Patent Application Publication
(10) Pub. No.: US 2011/0301104 A1
(43) Pub. Date: Dec. 8, 2011

(54) Transition state structure of orotate phosphoribosyl transferases and uses thereof

(76) Inventor: Vern L. Schramm, New Rochelle, NY (US)

(21) Appl. No.: 12/998,614

(22) PCT Filed: Nov. 18, 2009
(86) PCT No.: PCT/US2009/006171

§ 371 Date: Jun. 8, 2011

Related U.S. Application Data
(60) Provisional application No. 61/201,599, filed on Dec. 12, 2008.

(51) Int. Cl. A61K 31/7034; C12N 5/071; C12N 1/10; A61P 33/06; C07H 5/04; A61P 35/00; A61P 37/06; C12N 9/99; C12N 5/09

(52) U.S. Cl. 514/25; 435/184; 435/366; 435/258.2; 536/18.7

(57) ABSTRACT

Methods are provided for designing a transition state inhibitor of orotate phosphoribosyltransferase (OPRT) and for inhibiting OPRT.

### CROSS-REFERENCE TO RELATED APPLICATION

This application claims priority of U.S. Provisional Patent Application No. 61/201,599, filed on Dec. 12, 2008, the content of which is incorporated by reference.

### STATEMENT OF GOVERNMENT SUPPORT

The invention disclosed herein was made with U.S. Government support under National Institutes of Health Grant No. AI049512. Accordingly, the U.S. Government has certain rights in this invention.

### FIELD OF THE INVENTION

The present invention generally relates to transition state structures of orotate phosphoribosyltransferases (OPRTs), specifically for human and parasite enzymes, and their use for design of transition state inhibitors. Transition state analogue inhibitors of OPRT can be used to treat neoplastic and autoimmune diseases and malaria infections.

### BACKGROUND OF THE INVENTION

Throughout this application various publications are referred to in superscripts. Citations for these references may be found at the end of the specification immediately preceding the claims. The disclosures of these publications are hereby incorporated by reference in their entireties into the subject application to more fully describe the art to which the subject application pertains.

Plasmodium falciparum is a protozoan parasite that causes a severe form of human malaria. It is estimated that over one million people die annually of P. falciparum-related malaria with a majority under the age of five.¹ The development of new therapeutic approaches against malaria is urgent due to the increased resistance to current anti-malarial drugs.^{2,3} For P. falciparum and humans, de novo pyrimidine biosynthesis is the major pathway of nucleotide production. The viability of P. falciparum depends completely upon de novo pyrimidine biosynthesis, but mammals can also use a salvage pathway.⁴ P. falciparum does not encode enzymes of the pyrimidine salvage pathway.⁴⁻⁷ Inhibitors against de novo pyrimidine biosynthesis are fatal for the parasite.⁸⁻¹⁰

De novo pyrimidine biosynthesis requires six sequential reactions to generate uridine 5′-monophosphate (UMP).^{4,11} Orotate phosphoribosyltransferase (EC 2.4.2.10, OPRT) is the fifth enzyme and catalyzes the formation of orotidine 5′-monophosphate (OMP) from α-D-phosphoribosylpyrophosphate (PRPP) and orotate. OMP is subsequently converted to UMP by OMP decarboxylase (ODC). Given the essential function of UMP biosynthesis in P. falciparum, blocking OMP production may provide anti-malarial compounds.

OPRT homologues are widely distributed among bacteria, fungi, insect and mammals.¹² OPRT and ODC activities of most prokaryotes are located in two separately encoded enzymes. However, in higher eukaryotes both activities are associated within a single bifunctional protein, UMP synthase.^{4,13-19} Human UMP synthase (HsUMPase) contains N-terminal OPRT and C-terminal ODC domains.²⁰ It plays an essential role in the rapidly-proliferating cells, because these cells rely on de novo pyrimidine biosynthesis to satisfy the increased demand for nucleic acid synthesis.²¹ This feature has been successfully exploited in other inhibitors of de novo pyrimidine biosynthesis, including the antifolates. Anti-pyrimidine therapies are in use for the treatment of malignant neoplastic and autoimmune diseases.²²⁻²⁶ The human OPRT (HsOPRT) domain of HsUMPase provides another potential target for anti-cancer and immunosuppressive drugs.

Transition state theory predicts that transition state analogues bind more tightly to enzymes than substrates by the factor of catalytic rate acceleration. With typical accelerations of 10¹⁰-10¹⁵ and substrate affinity of 10⁻³-10⁻⁶ M, enzymes are expected to bind faithful mimics of transition state analogues with K_{d} values in the range of 10⁻¹⁴-10⁻²³ M.²⁷⁻³³ Transition state analogues can be designed from transition state models based on kinetic isotope effects (KIEs) interpreted through computational chemistry to match experimental and computed KIE values. Consequently, KIEs provide a powerful tool to access enzymatic transition state structures.^{30,32,34}

There is a need for improved treatments for malaria infections, malignancies and proliferative diseases, and autoimmune diseases. The present invention addresses that need.

### SUMMARY OF THE INVENTION

The invention provides a method for designing a transition state inhibitor of orotate phosphoribosyltransferase (OPRT) comprising designing a compound that resembles the charge and geometry of the OPRT transition state.

The invention also provides a method of inhibiting orotate phosphoribosyltransferase (OPRT) comprising designing a transition state inhibitor of OPRT by any of the methods disclosed herein and then contacting the OPRT with the inhibitor.

### BRIEF DESCRIPTION OF THE DRAWINGS

FIG. 1. Reaction catalyzed by PfOPRT and HsOPRT with phosphonoacetic acid (PA) as substrate.

FIG. 2. Synthesis of p-nitrophenyl β-D-ribose 5′-phosphate.

FIG. 3. Synthesis of isotopically labeled OMPs using [5′-¹⁴C]OMP as an example. [1′-¹⁴C]-, [¹′-³H]-, [2′-³H]-, [4′-³H]-, [5′-¹⁴C]-, [5′-³H₂]-, [1, 3-¹⁵N₂, 5′-¹⁴C]-, [3-¹⁵N, 5′-¹⁴C]- and [1, 3-¹⁵N₂, 1′-¹⁴C]OMP were prepared from isotopically labeled riboses, glucoses and orotates through one-pot, stepwise enzymatic reactions. The enzyme mixtures used for the synthesis contain ribokinase (RK), hexokinase (HK), glucose-6-phosphate dehydrogenase (G6PDH), phosphogluconic acid dehydrogenase (PGDH), L-glutamic dehydrogenase (GDH), phosphoriboisomerase (PRI), adenylate kinase (AK), pyruvate kinase (PK), phospho-D-ribosyl-1-pyrophosphate synthase (PRPPase) and orotate phosphoribosyltransferase (OPRT).

FIG. 4. Forward commitments to catalysis by PfOPRT and HsOPRT. Isotope trapping experiments were performed to measure the forward commitment factors. [1, 3-¹⁵N₂; 5′-¹⁴C]OMP was pre-incubated with OPRTs for 10 s and then rapidly mixed with chase buffers including cold OMP and various concentrations of PA. After five catalytic turnovers, the generated products were isolated by charcoal column chromatography and analyzed on the basis of the total radioactivity. The fractions of product formed vs bound substrate at time zero were plotted against PA concentrations. Forward commitment factors were calculated on the basis of

$${C}_{f} = \frac{{Y}_{ma x}}{1 - {Y}_{ma x}}$$

to give the C_{f} of PfOPRT (--∘--) of 0.035±0.006 and C_{f} of HsOPRT (ωωωΔωωω) of 0.042±0.003.

FIG. 5. Intrinsic KIEs for PfOPRT and HsOPRT. The positions for isotopic labeling are indicated in the substrate OMP. For each pair of KIE values, the upper value is for PfOPRT and the lower value is for HsOPRT.

FIG. 6. Transition state structures of PfOPRT and HsOPRT (Stick and MEP modes). The transition state structures were determined in vacuo by hybrid density functional theory implemented in Gaussian 98 using B3LYP functional and the 6-31 G (d, p) basis sets. The distances in the reaction coordinate (C1′-N1 and C1′-O^{PA}) for PfOPRT and HsOPRT transition states are shown. The molecular electrostatic potential (MEP) surfaces were generated by the CUBE subprogram of Gaussian 98 and are visualized using Molekel 4.0 at a density of 0.2 electron/Å³.

FIG. 7. Reaction coordinate for the conversion from OMP to the products orotate and 5-phosphoribosyl 1-phosphonoacetic acid (PRPA). The transition state structures of PfOPRT and HsOPRT are shown. MEP surfaces were generated by the CUBE subprogram of Gaussian 98 and visualized using Molekel 4.0 at a density of 0.2 electron/Å³.

### DETAILED DESCRIPTION OF THE INVENTION

The invention provides a method for designing a transition state inhibitor of orotate phosphoribosyltransferase (OPRT) comprising designing a compound that resembles the charge and geometry of the OPRT transition state.

The OPRT can have intrinsic kinetic isotope effects, for example, for [1′-¹⁴C], [1, 3-¹⁵N₂], [3-¹⁵N], [1′-³H], [2′-³H], [4′-³H] and [5′-³H₂] of 1.034, 1.028, 0.997, 1.261, 1.116, 0.974 and 1.013, respectively, or for [1′-¹⁴C], [1, 3-¹⁵N₂], [3-¹⁵N], [1′-³H], [2′-³H], [4′-³H] and [5′-³H₂] of 1.035, 1.025, 0.993, 1.199, 1.129, 0.962 and 1.019, respectively.

Preferably, the OPRT is a Plasmodium OPRT or a human OPRT.

The OPRT transition state can be characterized, for example, by one or more of, or preferably by two or more of, or by all of, a late dissociative D_{N}*A_{N}^{‡} transition state, a fully dissociated orotate, a ribooxacarbenium ion character, partially formed nucleophilic bonds, 2′-C-endo ribosyl geometry, and a C1′-O^{PA} distance of about 2.1 Å.

The method can comprise designing a chemically stable compound that resembles (a) the molecular electrostatic potential at the van der Walls surface computed from the molecular wave function of the transition state of OPRT and (b) the geometric atomic volume of the OPRT transition state.

As used herein, a compound resembles the OPRT transition state molecular electrostatic potential at the van der Walls surface computed from the molecular wave function of the transition state and the geometric atomic volume if that compound has an Sₑ and S_{g}≧0.5, where Sₑ and S_{g} are determined, for example, as in Formulas (1) and (2) on page 8831 of Bagdassarian et al., 1996.⁶²

Preferably, the compound comprises a moiety resembling the molecular electrostatic potential surface of the ribosyl group at the transition state.

The method can further comprises analyzing the compound for its ability to inhibit OPRT.

The invention also provides for the use of the orotate phosphoribosyltransferase (OPRT) transition state for designing an inhibitor of the OPRT transition state.

The invention also provides a method of inhibiting orotate phosphoribosyltransferase (OPRT) comprising designing a transition state inhibitor of OPRT by any of the methods disclosed herein and then contacting the OPRT with the inhibitor.

The OPRT can be in, for example, a parasite or a human cell. Preferably, the parasite is a protozoan parasite of the genus Plasmodium, such as, for example, Plasmodium falciparum, Plasmodium vivax, Plasmodium ovale or Plasmodium malariae.

The human cell can be, for example, a cancer cell or a cell in a subject suffering from an autoimmune disease. Autoimmune diseases include, but are not limited to, acute disseminated encephalomyelitis (ADEM), Addison's disease, alopecia greata, antiphospholipid antibody syndrome (APS), autoimmune hemolytic anemia, autoimmune hepatitis, bullous pemphigoid, coeliac disease, dermatomyositis, diabetes mellitus type 1, Goodpasture's syndrome, Graves' disease, Guillain-Barré syndrome (GBS), Hashimoto's disease, idiopathic thrombocytopenic purpura, lupus erythematosus, mixed connective tissue disease, multiple sclerosis, myasthenia gravis, pemphigus vulgaris, pernicious anaemia, polymyositis, primary biliary cirrhosis, rheumatoid arthritis, Sjögren's syndrome, Temporal arteritis, vasculitis, and Wegener's granulomatosis.

The present invention is illustrated in the following Experimental Details section, which is set forth to aid in the understanding of the invention, and should not be construed to limit in any way the scope of the invention as defined in the claims that follow thereafter.

### Experimental Details

### Introduction

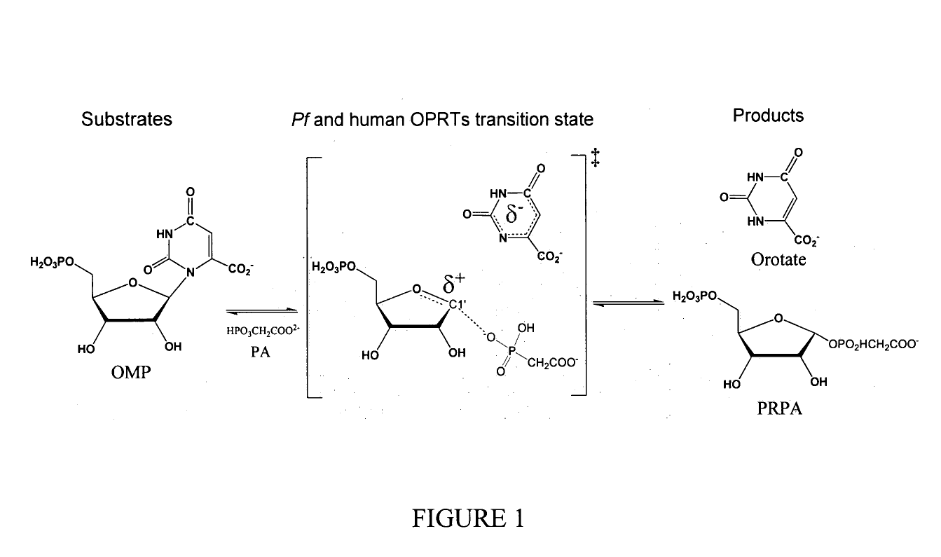

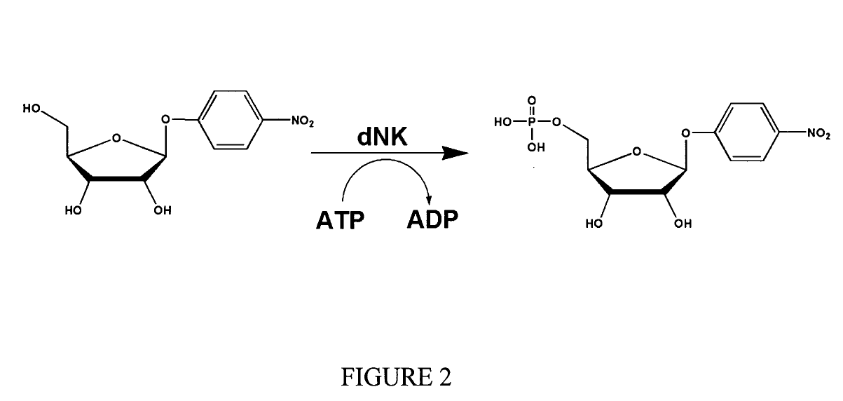

The intrinsic KIEs of PfOPRT and HsOPRT at geometry-sensitive positions were measured with phosphonoacetic acid (PA) as a slow substrate analogue of pyrophosphate and used as constraints for computational chemistry to explore their transition state structures (FIG. 1). The transition states are characterized by ribooxacarbenium ion character and fully dissociated orotate. Significant nucleophilic attack is involved at the OPRT transition states (D_{N}*A_{N}^{‡}). For enzymes with ribocations at their transition states, nitrophenylribosides provide mechanistic tools for distinguishing leaving group from ribocation activation.³⁵ p-Nitrophenyl β-D-ribose 5′-phosphate was synthesized with deoxyribonucleoside kinase (FIG. 2).³⁶ Poor catalytic activity with OPRTs indicate that leaving group activation is important for PfOPRT and HsOPRT. The D_{N}*A_{N}^{‡} transition states of PfOPRT and HsOPRT are different from the partially dissociative D_{N}^{‡}*A_{N} transition state of Salmonella typhimurium OPRT (StOPRT).³⁷ The OPRT transition states provide blueprints for the design of transition state analogues.

### Materials and Methods

Protein Expression Vector. The genes for PfOPRT and HsUMPase with a thrombin-cleavable His₆ tag at the N-terminal were synthesized chemically and cloned into the pDONR221 vector (DNA 2.0, Menlo Park, Calif.). Gene codons were optimized by adopting the commonly used codons (>10% usage frequency) in E. coli. The PfOPRT and HsUMPase genes were then recombined with the pDEST14 vector via LR Clonase II reaction (Invitrogen). The subsequent pDEST14-PfOPRT vector was used for protein expression. For the pDEST14-HsUMPase vector, a stop codon was introduced at L246 with QuikChange® II XL Site-Directed Mutagenesis Kits (La Jolla, Calif.). This HsUMPase truncation construct only expresses its HsOPRT domain. The plasmid genes were sequenced to confirm the integrity.

Expression and Purification of PfOPRT and HsOPRT. The pDEST14 vectors encoding the two OPRT genes were transformed into BL21-AI E. coli competent cells and grown in LB+Amp (100 μg mL⁻¹) at 37° C. to an OD₆₀₀ of 0.6. Protein expression was induced by adding 0.02% L-arabinose with overnight growth at 37° C. Cells were harvested by centrifugation at 4° C., weighted and resuspended in lysis buffer (20 mM Tris-HCl, 500 mM NaCl and 5 mM Imidazole, pH=7.9, 3 mL/gram cell pellet). Lysozyme (100 μg mL⁻¹), DNase I (10 μg mL⁻¹) and protease inhibitor (1 tablet/8 grams of cells) (Roche Applied Science, Indianapolis, Ind.) were added to the solution. Following disruption in a French Press, cell lysate was centrifuged at 30,000 g for 25 mM at 4° C. The supernatant was loaded onto a Ni²⁺ affinity column, washed with 5 column volume of binding buffer (the same as lysis buffer) and washed with 20 mM Tris-HCl, 500 mM NaCl and 20 mM imidazole, pH=7.9. The 6×His-OPRTs were eluted with a gradient of imidazole (20 mM ˜500 mM). The OPRT bands were visualized on SDS-PAGE gels and fractions with high purity were pooled, made to 10% glycerol and 10 mM β-ME (final concentration), concentrated and dialyzed overnight at 4° C. to remove imidazole. His-tags were removed during dialysis by the presence of thrombin (1 unit/0.1 mg). His-tag free OPRTs were purified by Superdex 200 26/60 Gel filtration column, concentrated and stored at −80° C. The enzyme activities were tested at 25° C. in mixtures containing 50 mM Tris-HCl, 5 mM MgCl₂, pH=8.0 and varied concentrations of OMP and pyrophosphate. Reactions were monitored by the absorbance increase at 295 nm to give an extinction coefficient of 3.67 mM⁻¹ cm⁻¹.

Reagents and Materials. D-[6-³H₂]glucose-6-phosphate, D[5-³H]glucose, [6-¹⁴C]glucose, D[1-³H]ribose and D[1-¹⁴C]ribose were purchased from American Radiolabeled Chemicals Inc. [1, 3-¹⁵N₂]orotic acid and [¹⁵N₂]urea were purchased from Cambridge Isotope Laboratories. p-Nitrophenyl β-D-ribose was generously provided by Dr. Peter C. Tyler (Industrial Research Ltd., New Zealand). [2-³H]Ribose was synthesized as described previously.³⁸⁻⁴⁰ Hexokinase (HK), glucose 6-phosphate dehydrogenase (G6PDH), phosphogluconic acid dehydrogenase (PGDH), L-glutamic acid dehydrogenase (GDH), phosphoriboisomerase (PRI), adenylate kinase (AK) and pyruvate kinase (PK) were purchased from Sigma. Phospho-D-ribosyl-α-1-pyrophosphate synthase (PRPPase) and ribokinase (RK) were prepared as described before.^{41,42} Deoxynucleoside kinase from Anopheles gambiae (Ag-dNK) was kindly provided by Dr. Maria B. Cassera (Department of Biochemistry, Albert Einstein College of Medicine). All other reagents were purchased from readily available commercial sources and used without further purification. Nitrogen nuclear magnetic resonance (¹⁵N NMR) was measured in DMSO-d₆ using a Bruker 300 MHz instrument. The ¹⁵N NMR spectra were collected using continuous proton-decoupling mode with at least 10000 scans. Mass spectra were acquired on an IonSpec FT-ICR mass spectrometer (Varian Inc., CA).

Synthesis of [3-¹⁵N]orotic acid. [3-¹⁵N]Orotic acid was synthesized based on previous reports with some modifications.^{43,44} Briefly, 1.47 g of DL-aspartic acid, 1.74 g of KOH and 0.66 g of [¹⁵N₂]urea were dissolved in 33.3 mL of water. The mixture was refluxed at 115° C. for 2 hr, then two portions of 0.66 g of [¹⁵N₂]urea were added separately with a 2 hr interval. After 6 hr reaction, the mixture was acidified with 3 mL of concentrated HCl and allowed to stand overnight at room temperature. Precipitated ureidosuccinic acid was filtrated and recrystallized from water. Ureidosuccinic acid (0.82 g) was dissolved in 2.73 mL of 20% HCl. The mixture was evaporated to dryness on a hot plate. The resulting 5-acetic acid-hydantoin was recrystallized from water. Crystals (0.62 g) were mixed with 2.46 mL of glacial acetic acid and 0.62 g of bromine and heated at 100° C. in a sealed tube for 2 hr. Precipitated 5-carboxymethylidine-hydantoin was filtrated, recrystallized from water and 0.42 g of the product was added to 14 mL of 1 M KOH. Following 64° C. for 2 hr, the [3-¹⁵N]orotic acid was precipitated by acidification with concentrated HCl and recrystallization from water. [3-¹⁵N]orotic acid: ¹⁵N NMR (75 MHz, DMSO-d₆) δ ppm 161; m/z std C₅H₅N₂O₄ (M+1) 157.026, C₅H₅¹⁵N₁N₁O₄ (M+1) 158.023.

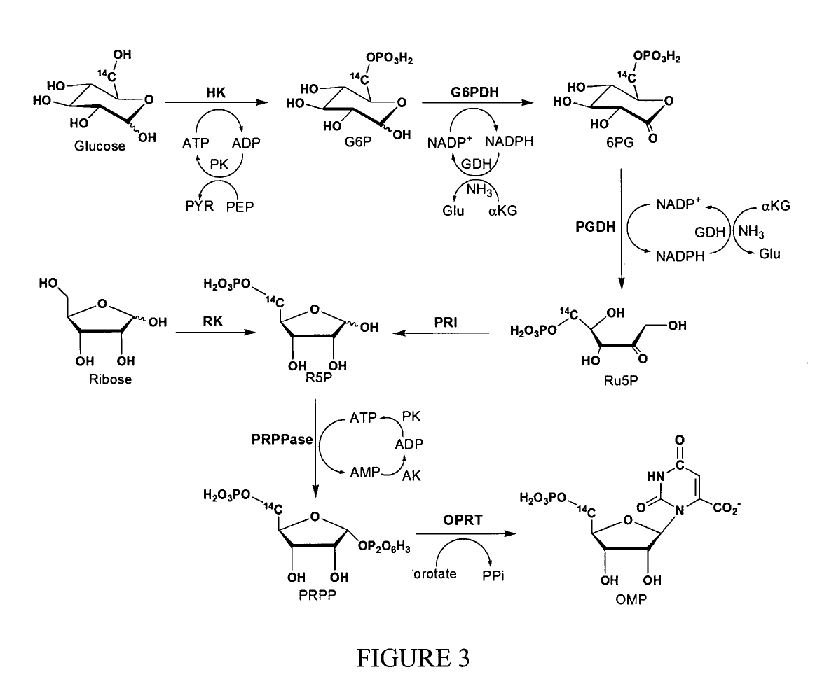

Synthesis of Isotopically Labeled OMPs. [1′-³H]OMP, [1′-¹⁴C]OMP and [2′-³H]OMP were prepared enzymatically from [1-³H]ribose, [1-¹⁴C]ribose and [2-³H]ribose, respectively. [4′-³H]OMP, [5′-³H₂]OMP and [5′-¹⁴C]OMP were synthesized from [5-³H]glucose, [6-³H₂]glucose-6-phosphate and [6-¹⁴C]glucose, respectively. [3-¹⁵N]OMP and [1, 3-¹⁵N₂]OMP were synthesized with [3-¹⁵N]- and [1, 3-¹⁵N₂]orotic acid as precursors, respectively (e.g. FIG. 3).

Synthesis of OMP from ribose and orotate used a reaction mixture with 2.5 mM orotate, 1 mM ribose, 100 mM phosphate (pH=7.4), 50 mM glycylglycine, 50 mM KCl, 20 mM MgCl₂, 20 mM phosphoenolpyruvate, 1 mM ATP and 2 mM DTT. The reactions were initialized by adding an enzyme mixture containing 1 unit AK, 1 unit PK, 0.01 unit RK, 0.008 unit PRPPase and 0.033 unit PfOPRT. This reaction mixture was incubated at 37° C. for 20 hr to reach completion. OMP products were then purified twice by reverse phase HPLC (C-18 Deltapak column, 20 mM KH₂PO₄, 8 mM tetrabutylammonium, pH=6.0 as eluting buffer). The OMPs were concentrated and repurified by HPLC, eluted with 2% (v/v) MeOH/H₂O. The overall yields of OMP varied from 40% and 90% with reference to ribose.

For OMP synthesis from glucose, the reactions contained 2.5 mM orotate, 1 mM glucose, 100 mM phosphate, 50 mM glycylglycine, 50 mM KCl, 20 mM MgCl₂, 5 mM NH₄C1, 20 mM phosphoenolpyruvate, 1 mM ATP, 0.1 mM NADP⁺, 20 mM α-keto glutarate and 2 mM DTT (pH=7.4). The reaction was initialized by adding 1 unit HK, 1 unit G6PDH, 0.075 unit PGDH, 0.02 unit PRI, 1 unit GDH, 0.016 unit PRPPase, 0.033 unit PfOPRT, 1 unit AK and 1 unit PK (pre-mixed enzyme solution). The reaction mixture was then incubated at 37° C. for 20 hr to generate OMP. The OMP products were purified as described above. The overall yields of OMP were 30% to 70% from starting glucose. After the last step of HPLC purification (described above), isotopically labeled OMPs were lyophilized, dissolved in H₂O and stored at −80° C. for further use.

Determination of Kinetic Isotope Effects. KIEs were measured under competitive conditions using the isotope ratio method. Briefly, the ³H- and ¹⁴C-labeled OMPs were mixed at a ratio of 4:1 (cpm) with at least 5×10⁴ cpm for ¹⁴C. The OMP mixture was then added into a solution containing 50 mM Tris-HCl (pH=8.0), 5 mM MgCl₂, 20 mM PA and 100 μM OMP (labeled and unlabeled). All the experiments were performed at 25° C. with 1.2 mL reaction volume in the presence of either 0.7 μM PfOPRT or 0.9 μM HsOPRT. Part of the reaction mixture (240 μL) was allowed to proceed to 100% completion by adding another 4.5 μM PfOPRT (final concentration) and incubating an additional 4 hr. The rest of the reaction was stopped at 20 to 30% conversion to product. The two reaction mixtures (100% and 20-30%) were loaded onto charcoal-cellulose columns (1:4 w/w, pre-equilibrated with H₂O), followed by washing with 1 mL H₂O. The isotopically labeled reaction products were then eluted with 3 mL 3.3% (v/v) NH₄OH/H₂O (pH=6.6). The 3 mL fractions were dried by speedvac, mixed with 200 μL H₂O and then 10 mL scintillation counter cocktail (Ultima Gold) as described before.^{39,40} These samples were counted for at least 8 cycles to give ³H:¹⁴C ratios (20 min/cycle, Wallac 1414 LSC, PerkinElmer) and averaged. For each KIE measurement at least 5 replicates were performed.

The ¹⁴C channel ratio was calculated by determining the ratio between channel 1 and channel 2 through counting the [1-¹⁴C]ribose standard. The channel 1 and 2 were set to keep all ³H signals in channel 1, whereas only ¹⁴C counts appear in channel 2. The following equations were used for data processing:

cpm(³H)=cpm_{channel 1}−cpm_{channel 2}×(¹⁴C channel ratio), and cpm(¹⁴C)=cpm_{channel 2}×(1+¹⁴C channel ratio).

The ³H:¹⁴C ratio was determined for the partial and complete conversion. The KIEs were corrected to 0% reaction based on the eq. 1, where f is the fraction of the reaction completion, R_{f} and R₀ are the ratios of the heavy isotope to the light isotope at the partial and complete conversion, respectively.

$$KIE = \frac{ln ( 1 - f )}{ln ( 1 - f \times \frac{Rf}{{R}_{0 }} )} \qquad ( 1 )$$

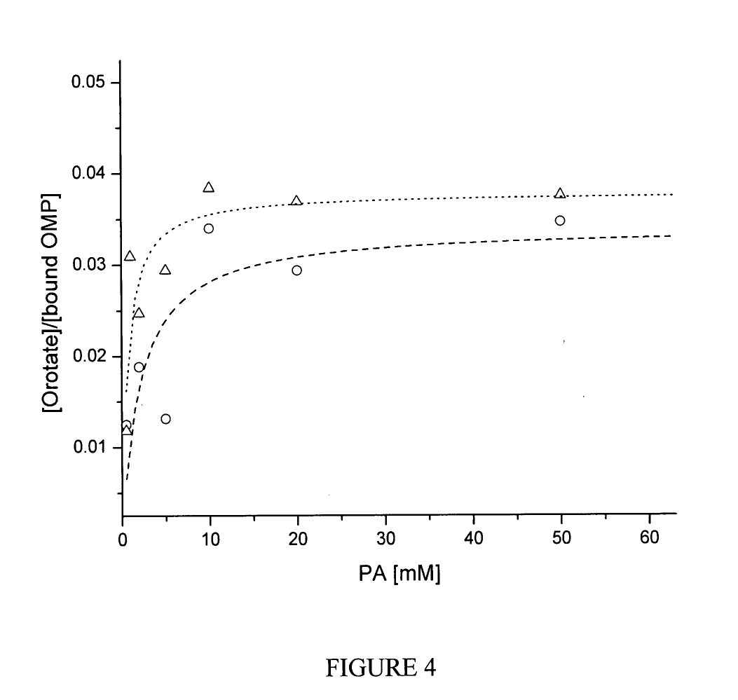

Measurement of Forward Commitment Factor. The forward commitment factors of OPRT-catalyzed reactions were measured by isotope trapping enzyme-bound radiolabeled substrate in the presence of excessive unlabeled substrate.^{39,40,45,46} Briefly, 31 μM OPRT was pre-incubated with 60 μM [1, 3-¹⁵N₂, 5′-¹⁴C]OMP for 10 s at 25° C. in a total volume of 10 μL solution (50 mM Tris-HCl, 5 mM MgCl₂ and pH=8.0). The reaction mixture was rapidly chased with 140 μL buffer containing 50 mM Tris-HCL (pH=8.0), 5 mM MgCl₂, 5 mM unlabeled OMP and various concentrations of PA. After five catalytic turnovers, the reaction mixture was loaded onto the charcoal:cellulose column (1:4, w/w) pre-equilibrated with H₂O, followed by washing with 1 mL H₂O. Products were eluted with 3 mL 3.3% (v/v) NH₄OH/H₂O (pH=6.6) and the radioactivity of products were determined by liquid scintillation counting. Control experiments in the absence of enzyme provided background correction. The specific radioactivity of labeled product versus bound substrate in the Michaelis complex was plotted against PA concentrations (FIG. 4). The forward commitment factors were calculated on the basis of the equation

$${C}_{f} = \frac{{Y}_{ma x}}{1 - {Y}_{ma x}} ,$$

where Yₘₐₓ is the y axial intercept upon extrapolating the plateau region and C_{f} is forward commitment factor.

Computational Modeling of Transition State Structures. The transition state structures were determined in vacuo by hybrid density functional theory implemented in Gaussian 98.⁴⁷ The structures of OMP and the 5′-phosphoribosyl oxacarbenium ion containing partly or fully dissociated orotate in different ionization states and with varied extents of PA nucleophilic participation were optimized using B3LYP functional and 6-31G (d, p) basis sets. To match the intrinsic KIEs obtained experimentally, the bond distances of C1′-N1 and C1′-O^{PA} were varied systematically. The bond frequencies of substrate and transition states were calculated at the same level of theory and basis sets. Computational KIEs were obtained on the basis of bond frequency changes from substrate to transition states at the temperature of 298° K using the ISOEFF98 program.⁴⁸ All 3N-6 vibrational modes were used for calculation. The bond frequencies of substrate, transition state and a reaction-coordinate imaginary frequency of 50i cm⁻¹ (or greater) were retrieved as input files to calculate the isotope effects. The transition state structures were iteratively altered and optimized until the calculated KIEs provided a best match to the intrinsic KIEs.

Calculation of Molecular Electrostatic Potential Surfaces. The molecular electrostatic potential (MEP) surfaces were calculated by employing the CUBE program in Gaussian 98. The checkpoint files for CUBE inputs were generated during geometry optimization at the B3LYP/6-31G (d, p) level. The MEP surfaces were visualized by Molekel 4.0 at a density of 0.2 electron/Å³.^{49,50}

Synthesis of p-nitrophenyl β-D-ribose 5′ phosphate and enzymatic activity assay. p-Nitrophenyl β-D-ribose 5′-phosphate was synthesized by 5′-phosphorylation of p-nitrophenyl β-D-ribose by Ag-dNK. The reaction mixtures contained 50 mM Tris-HCl (pH=8.0), 5 mM MgCl₂, 50 mM KCl, 0.2 mM DTT, 8 mM ATP, 4 mM p-nitrophenyl β-D-ribose and 7 μM Ag-dNK. After overnight incubation at 37° C., p-nitrophenyl β-D-ribose 5′-phosphate was purified by reverse phase HPLC (C-18 Deltapak column) with a gradient of acetonitrile from 3% to 30% in a buffer containing 100 mM KH₂PO₄ (pH=6.0) and 8 mM tetrabutylammonium. p-Nitrophenyl β-D-ribose 5′-phosphate was then concentrated by rotovap, repurified by HPLC using elution with methanol from 2% to 50% in H₂O to provide a 28% overall yield, concentrated and stored at −80° C. The p-nitrophenyl 13-D-ribose 5′-phosphate: m/z C₁₁H₁₃NO₁₀P (M−1) 350.029 observed; 350.028 calculated.

The PfOPRT and HsOPRT catalytic activities with p-nitrophenyl β-D-ribose 5′-phosphate were measured at 25° C. in solutions containing 50 mM Tris-HCl (pH=8.0), 5 mM MgCl₂ and varied concentrations of p-nitrophenyl β-D-ribose 5′-phosphate and PA or pyrophosphate. Reaction rates were monitored through the absorbance increase at 400 nm.

### Results and Discussion

Experimental KIEs, Commitment factor and Intrinsic KIEs. The experimental KIEs were measured under competitive conditions. The 5′-¹⁴C of OMP is four bonds away from the reaction center and the [5′-¹⁴C] KIE is assumed to be unity. The observed KIEs of [1′-³H], [2′-³H], [4′-³H] and [5′-³H₂] were determined with [5′-¹⁴C]OMP as the silent remote label. The KIEs of [1′-¹⁴C], [1, 3-¹⁵N₂, 5′-¹⁴C], [3-¹⁵N, 5′-¹⁴C] and [1, 3-¹⁵N₂, 1′-¹⁴C] were obtained with reference to [4′-³H]OMP as the remote label. These observed KIEs were then corrected for the corresponding KIE of the remote label ([4′-³H]) to give experimental KIEs.

The experimental KIEs contained the contribution from the commitment to catalysis (commitment factors), which can mask intrinsic KIEs under some circumstances.^{39,40} However, the commitment factors (forward and reverse) on intrinsic KIEs are within experimental error limits for PfOPRT and HsOPRT with PA as a slow substrate. The forward commitment factors of 0.035 for PfOPRT and 0.042 for HsOPRT were applied in the equation ^{x}(V/K)=(^{x}k+C_{f})/(1+C_{f}) where ^{x}(V/K) is the experimental KIE, ^{x}k is the intrinsic KIE and C_{f} is the forward commitment factor.⁵¹ The experimental ^{x}(V/K) and the intrinsic KIE (^{x}k) were the same within experimental error (FIG. 4 and Table 1). This result is expected for PA as slow substrate with a small k_{cat} of 0.023s⁻¹ for PfOPRT and 0.017 s⁻¹ for HsOPRT, and a large Kₘ of 1.59 mM for PfOPRT and 3.84 mM for HsOPRT. In cases where k_{cat}/Kₘ is small, forward commitment is minimized.^{39,40,45} PfOPRT and HsOPRT display large [1′-³H] ^{T} (V/K) KIEs and are near the theoretical maxima for [1, 3-¹⁵N₂] ^{N}(V/K) KIEs. These values support a dissociated orotate (Table 1 and discussion below). Large KIE values also indicate that the reverse commitments of PfOPRT and HsOPRT have negligible effects on intrinsic KIE values.^{41,52-54} Significant reverse commitment causes experimental KIE ^{x}(V/K) to be smaller than intrinsic ^{x}k. Experimental KIEs shown in Table 1 are therefore intrinsic KIEs for PfOPRT and HsOPRT.

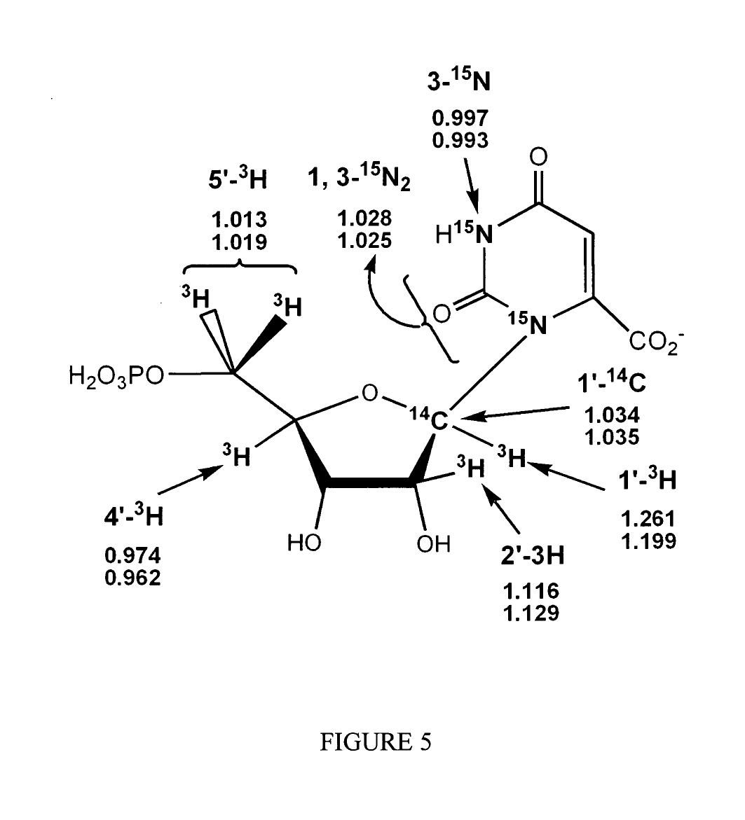

Modeling Transition States. Transition state structures were estimated by computational modeling with experimental intrinsic KIEs as constraints. All calculations were carried out in vacuo at the B3LYP/6-31G (d, p) level. The geometry of the substrate OMP was first optimized without constraints and this geometry was maintained for transition state analysis. Large [1′-³H] KIEs around 20% combined with modest [1′-¹⁴C] KIEs of 3.5% are indicative of ribocationic transition state features with weak bonding to C1′ (FIG. 5). Consequently, the transition state optimization started with a 5′-phosphoribosyl oxacarbenium ion containing weak bonding to orotate or to PA. Bond distances between 1.8 Å to 2.6 Å were searched for C1′-N1 and C1′-O^{PA} to match intrinsic [1′-¹⁴C] KIE and [1, 3-¹⁵N₂] KIE for PfOPRT and HsOPRT. Only transition state structures with the C1′-N1 distances ≧2.6 Å agreed with the [1, 3-¹⁵N₂] KIEs. The C1′-O^{PA} bond distances in the range of 2.14˜2.15 Å are consistent with [1′-¹⁴C], [1′-³H] and [2′-³H] KIEs (Table 1).

Geometries at the 4′- and 5′-regions of the transition states were optimized to match [4′-³H] and [5′-³H₂] intrinsic KIEs. OPRT crystal structures with OMP suggest geometry for the OMP 5′-regions.^{55,56} These 5′-conformations were optimized using constraints to match experimental [4′-³H] and [5′-³H₂] KIEs.

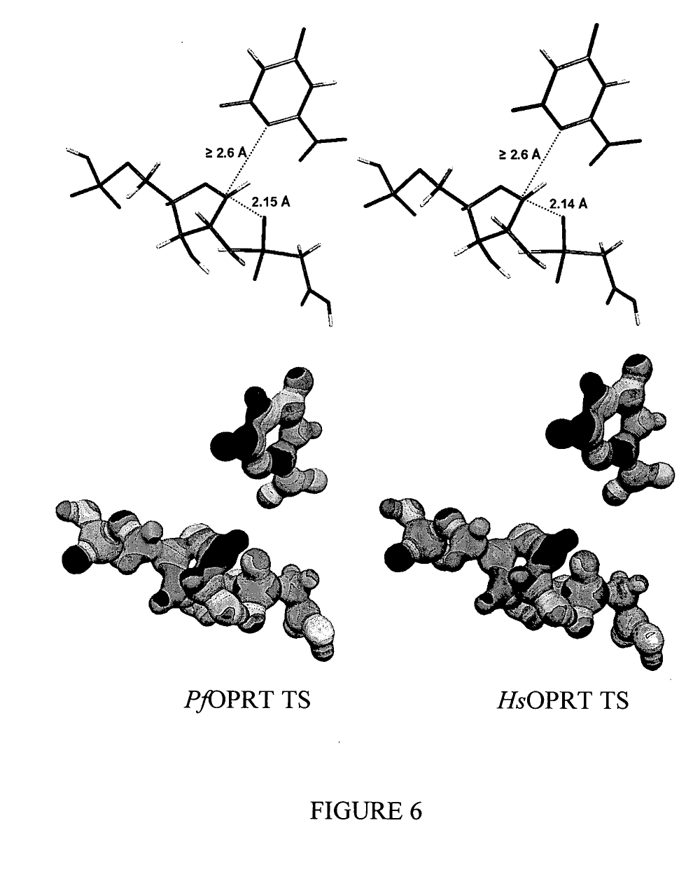

Transition State Structures of PfOPRT and HsOPRT. Alignment between PfOPRT and HsOPRT indicated that they share only 26% identity in amino acid sequence. However, the intrinsic KIEs indicate that their transition states are similar in regard to orotate dissociation, the nature of their oxacarbenium ions at the transition state, nucleophilic participation and ribosyl conformation. The intrinsic KIEs for [1′-³H], [2′-³H], [4′-³H], [5′-³H_{2], [}1′-¹⁴C], [1, 3-¹⁵N₂, 5′-¹⁴C], [3-¹⁵N, 5′-¹⁴C] and [1, 3-¹⁵N₂, 1′-¹⁴C] serve as boundary conditions to define the transition state structures of PfOPRT and HsOPRT (Table 1). The PfOPRT and HsOPRT transition state structures both display late associative D_{N}*A_{N}^{‡} characteristics. This transition state is developed after formation of a ribooxacarbenium species with fully dissociated orotate and partial bonding to the attacking PA. The orotates at these transition states are at least 2.6 Å distant from the anomeric carbons (FIG. 6). The characteristic ribooxacarbenium ions show a C1′-O^{PA} distance of 2.15 Å for PfOPRT and 2.14 Å for HsOPRT transition states. These distances are derived from the primary [1′-¹⁴C] and [1, 3-¹⁵N₂] intrinsic KIEs by matching calculated KIEs (Table 1). In the PfOPRT and HsOPRT transition state structures, the ribosyl rings both adopt 2′-C-endo conformations, giving a calculated [2′-³H] KIEs of 14%, consistent with the observed KIEs of 12 to 13%. The 5′-regions of the transition states with a C3′-C4′-05′-05′ dihedral angle of −147° are consistent with the [4′-³H] and [5′-³H₂] KIEs.

The late D_{N}*A_{N}^{‡} transition states of PfOPRT and HsOPRT are distinct from other N-ribosyltransferase enzymes. Most purine N-ribosyltransferase transition state structures have neutral leaving groups resulting from N7 protonation. In the PfOPRT and HsOPRT transition states, the leaving group orotates appear anionic as indicated by the leaving group ¹⁵N KIE values discussed below.

[1, 3-¹⁵N₂] and [3-¹⁵N] KIEs. The [1, 3-¹⁵N₂] and [3-¹⁵N] KIEs of PfOPRT and HsOPRT arise from the extent of C1′-N1 bond dissociation as well as leaving group interactions of orotate at the transition states. Without protonation at the transition states, the calculated [1-¹⁵N] KIE for a fully dissociated orotate dianion is 1.021, near the theoretical maximum. The [3-¹⁵N] contributes an additional KIE of 1.004 to give the theoretical [1, 3-¹⁵N₂] KIE of 1.025, consistent with the experimental [1, 3-¹⁵N₂] KIEs of 1.028 for PfOPRT and 1.025 for HsOPRT. The calculated [3-¹⁵N] KIE of 1.004 for dissociated orotate agrees with the intrinsic [3-¹⁵N] KIEs of 0.997 for PfOPRT and 0.993 for HsOPRT. Transition state modeling indicated that protonation at O2, O4 or O7 (the exocyclic carboxyl group) causes the expected [1, 3-¹⁵N₂] and [3-¹⁵N] KIEs to be inconsistent with experimental values (Table 2). On the basis of the intrinsic [1, 3-¹⁵N₂] and [3-¹⁵N] KIEs, the PfOPRT and HsOPRT transition states display full loss of the C1′-N1 bond order to generate dianionic orotates. Despite the apparent lack of protonation of the leaving group, the anionic charge is likely to be stabilized by multiple hydrogen bond or ion-pair interactions at the catalytic site. Isotope effects are most sensitive to atomic bond changes and these electrostatic interactions are not likely to be represented in the KIEs.

Full loss of the C1′-N1 bond at the PfOPRT and HsOPRT transition states is further confirmed by the large [2′-³H] and [1′-³H] KIEs (discussion below). Since C1′-N1 distances greater than 2.6 Å cause no additional increase in [1, 3-¹⁵N₂] KIEs, the C1′-N1 distances at these transition states are at least 2.6 Å (FIG. 6).

[1′-¹⁴C] and [1′-¹⁴C; 1, 3-¹⁵N₂] Primary KIEs. The primary [1′-¹⁴C] KIE of OPRT is sensitive to motion along the reaction coordinate and also reflects the interactions of leaving group and nucleophile with the anomeric carbon. Thus, [1′-¹⁴C] KIEs are often key parameters for determining mechanisms (S_{N}1 or S_{N}2) of N-ribosyltransferase enzymes.⁵⁷ A unity or slightly inverse [1′-¹⁴C] KIE indicates a fully dissociative transition state (D_{N}*A_{N}) with an isolated ribooxacarbenium ion structure.⁴¹ For transition states with weak to modest participation of leaving group or nucleophile, [1′-¹⁴C] KIEs are in the range of 1.01˜1.06.⁴¹ The degree of participation for either leaving group or nucleophile influences the values of intrinsic [1, 3-¹⁵N₂], [1′-³H] and [2′-³H] KIEs.^{45,52,53}

The intrinsic [1′-¹⁴C] KIEs of 1.034 for PfOPRT and 1.035 for HsOPRT are indicative of either early dissociative (D_{N}^{‡}*A_{N}) or late associative (D_{N}*A_{N}^{‡}) mechanisms (FIG. 5 and Table 1).^{45,52-54} Transition state candidates were optimized with systematically altered C1′-N1 and C1′-O^{PA} distances. Early dissociative (D_{N}^{‡}*A_{N}) structures were ruled out on the basis of unmatched [1, 3-¹⁵N₂] KIEs (see the discussion above). Although several other structures exhibit similar calculated and intrinsic [1′-¹⁴C] KIE values, only two structures could match [1′-¹⁴C] KIEs as well as [1′-³H] and [2′-³H] KIEs (results below).

The C1′-O^{PA} bond distances of 2.15 Å for the PfOPRT and 2.14 Å for the HsOPRT transition states gave the best agreement with the intrinsic KIEs (FIG. 6). These distances were consistent with the [1′-¹⁴C] KIEs of 1.034 and 1.035 and the large [1′-³H] KIEs of 1.199 and 1.261. Formation of the ribooxacarbenium ion transition state accounts for the large [1′-³H] KIE, while participation of the nucleophile gives rise to the significant [1′-¹⁴C] KIEs resulting from the C1′-O^{PA} bond order.

To support the primary [1′-¹⁴C] and [1, 3-¹⁵N₂] KIE measurements the combined [1′-¹⁴C, 1, 3-¹⁵N₂] KIEs were measured. The intrinsic [1′-¹⁴C; 1, 3-¹⁵N₂] KIE of 1.068 for PfOPRT is consistent with the product (1.063) of the [1′-¹⁴C] KIE of 1.034 and [1, 3-¹⁵N₂] KIE of 1.028. For HsOPRT, the intrinsic [1′-¹⁴C; 1, 3-¹⁵N₂] KIE of 1.076 also agrees with the product of the [1′-¹⁴C] and [1, 3-¹⁵N₂] KIEs within experimental errors (Table 1), supporting the accuracy of individual [1′-¹⁴C] and [1, 3-¹⁵N₂] KIEs used for transition state analysis.

[1′-³H] α-secondary KIEs. The [1′-³H] KIE originates primarily from the new out-of-plane mode as C1′ rehybridizes from sp³ to sp² at the transition state. Small [1′-³H] KIEs in N-ribosyltransferases suggest nucleophilic substitutions (S_{N}2) or partially dissociated D_{N}^{‡}*A_{N} transition states, while large [1′-³H] KIEs are indicative of fully dissociated S_{N}1 or partially dissociated D_{N}*A_{N}^{‡} transition states. The new out-of-plane bending mode for the C1′-H1′ bond as sp³ to sp² rehybridization occurs at the transition state increases the magnitude of the [1′-³H] KIE. However, a restricted stretching mode for the C1′-H1′ bond at the transition state decreases the [1′-³H] KIEs. The out-of-plane bending mode is the more dominant effect.⁵⁸ The bending mode is sensitive to the variations of C1′-N1 bond distances and the participation of nucleophile. PfOPRT and HsOPRT show large intrinsic [1′-³H] KIEs of 1.261 and 1.199, respectively (FIG. 5). The magnitudes of the [1′-³H] KIEs are consistent with full loss of the orotate dianion at the transition states, where the out-of-plane bending motions of ribooxacarbenium intermediate contribute strongly to the observed [1′-³H] KIEs. Although calculated [1′-³H] KIEs of 1.335 for PfOPRT and 1.330 for HsOPRT are substantially larger than the experimental KIEs (Table 1), it has been documented that [1′-³H] KIEs are often overestimated by in vacuo computation due to condensed-phase uncertainty around the C1′-H1′ region.^{46,52,59} Constraints from van der Waals interactions at the active sites are proposed to suppress the [1′-³H] KIEs.

[2′-³H] β-secondary KIEs. The [2′-³H] KIE values are informative for ribosyl geometry, leaving group dissociation and nucleophilic participation at the transition states. They arise from hyperconjugation (orbital overlap) of the C2′-H2′ σ bond to the vacant 2p_{z} orbital of the anomeric carbon and the occupancy of the 2p_{z} orbital. Occupancy of 2p_{z} also depends on the extent of C1′-N1 dissociation and C1′-O^{PA} participation at the transition states. The intrinsic 2′-³H KIEs of PfOPRT and HsOPRT of 1.116 and 1.129, respectively, are similar to the calculated [2′-³H] KIEs of 1.142 for PfOPRT and 1.140 for HsOPRT (Table 1) for full dissociation of orotate, modest participation of the attacking nucleophile and 2′-C-endo ribosyl geometry (see below).

The large [2′-³H] KIEs of PfOPRT and HsOPRT rule out early transition states with partial bonds to orotate (D_{N}^{‡}*A_{N}). Computational modeling indicates small [2′-³H] KIEs for early transition states with partially occupied 2p, orbitals. The large [2′-³H] KIEs of PfOPRT and HsOPRT also indicate ribosyl transition states with 2′-C-endo geometry and a H1′-C1′-C2′-H2′ dihedral angles of 71° for PfOPRT and 70° for HsOPRT (FIG. 6). In contrast, 2′-C-exo transition states give smaller [2′-³H] KIEs, where the H1′-C1′-C2′-H2′ dihedral angle of 2′-C-exo conformers is close to zero and thus compromises C2′-H2′→C1′-2p_{z} hyperconjugation.

[5′-³H₂] Remote KIEs. The intrinsic [5′-³H₂] KIEs are 1.013 for PfOPRT and 1.019 for HsOPRT (Table 1) even through 5′-³H₂ are four bonds distant from C1′ and not expected be influenced by chemistry at the reaction center. These modest [5′-³H₂] KIEs reflect remote conformational changes at the 5′-regions in transition state formation and arise from the product of the 5′-H-proR and 5′-H-proS KIEs. Computational studies on the 5′-conformation were accomplished by constraints selected from the OPRT crystal structures containing OMP molecules.^{55,56} Optimized structures with a restricted O4′-C4′-C5′-5′-H-proR dihedral angle of −137° gave consistent [5′-³H₂] KIE values for PfOPRT and HsOPRT (Table 1). Remote KIEs at the 5′-regions indicates bond distortion remote from chemistry imposed by the enzyme catalytic sites.

[4′-³H] Remote KIEs. The intrinsic [4′-³H] KIEs are 0.974 for PfOPRT and 0.962 for HsOPRT (FIG. 5). The [4′-³H] KIE is influenced by hyperconjugation between the C4′-H4′ σ* antibonding orbital and the O4′ long pair.⁵² An oxacarbenium ion transition state causes the O4′ nₚ electrons to redistribute toward the positively-charged anomeric carbon, causing decreased hyperconjugation between the C4′-H4′ σ* antibonding orbital and the O4′ n, electrons. A shorter C4′-H4′ bond at the transition state relative to the substrate causes inverse [4′-³H] KIEs (Table 3). The [4′-³H] KIE is also influenced by polarization of the 3′-hydroxyl group, which causes normal [4′-³H] KIEs.^{53,54} The [4′-³H] KIEs of the solved PfOPRT and HsOPRT transition states were calculated to be 0.972, which agree with the intrinsic [4′-³H] KIEs of 0.962 and 0.974. The small inverse [4′-³H] KIEs of PfOPRT and HsOPRT are consistent with a well-developed ribocation transition state. The inverse [4′-³H] KIEs indicate that the 3′-hydroxyl groups are not polarized in PfOPRT and HsOPRT transition states, as they are in some N-ribosyltransferases.^{52,54}

Kinetic Constants. With OMP and PA as a slow substrate analogue for PPi, the catalytic efficiency (k_{cat}/Kₘ) for PfOPRT and HsOPRT were approximately equivalent at 4.8×10³ and 7.9×10³ M⁻¹ s⁻¹, respectively. These values are reduced 110-fold for PfOPRT and 8.3-fold for HsOPRT relative to their (k_{cat}/Kₘ) values with PPi as the nucleophile (Table 4). These catalytic reductions are sufficient to give near-intrinsic KIE values as described above. The differences are dominated by k_{cat} changes, and as the Kₘ values are all in the range of 1 to 4 μM, relatively tight binding as expected for intermediates in a linear, essential biosynthetic pathway.

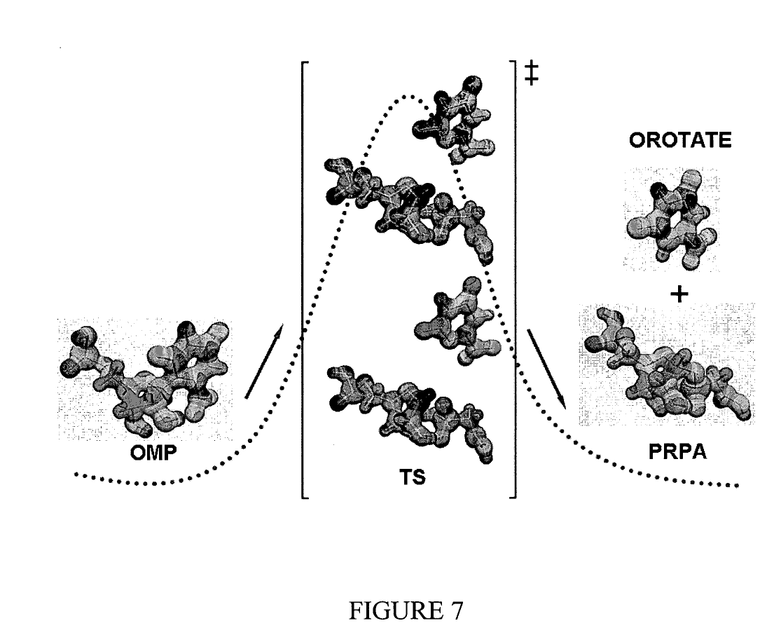

Leaving Group Activation. PfOPRT and HsOPRT could achieve their oxacarbenium ion transition states through leaving group activation, oxacarbenium ion formation or 2′-hydroxyl ionization (FIG. 7). p-Nitrophenyl β-D-ribose 5′-phosphate was used to distinguish leaving group activation from mechanisms of ribooxacarbenium ion formation and/or stabilization. If the enzyme activates the substrate through ribosyl activation, the p-nitrophenyl group provides an excellent leaving group, needing no enzymatic assistance, and p-nitrophenyl β-D-ribose 5′-phosphate would be an excellent substrate.³⁵ Assays indicated that PfOPRT did not catalyze the O-glycosidic bond cleavage of p-nitrophenyl β-D-ribose 5′-phosphate under conditions that could detect 10⁻⁶ of normal activity. Poor catalytic activity of PfOPRT and HsOPRT for the p-nitrophenyl β-D-ribose 5′-phosphate establishes that leaving group activation is a major force in OPRT reactions, therefore, base recognition plays an important catalytic role. The intrinsic KIEs for PfOPRT and HsOPRT indicated that orotate leaving group is not activated through protonation of any single group. However, leaving group activation may be accomplished through multiple hydrogen bond and/or ionic interactions with active site residues. Crystal structures of the yeast OPRT in complex with orotate, PRPP and Mg²⁺ ion support this proposal with orotate O4 in H-bond distance with a peptide bond NH and a guanidinium ion, N3 in contact with a peptide bond oxygen and the carboxylate hydrogen bonded to a threonine sidechain, a peptide NH and two ordered water molecules. Likewise, O2 of orotate also contacts two ordered water molecules.⁵⁶

Nitrophenyl Ribosides as Inhibitors of OPRTs. Lack of catalytic activity with substrate analogues can also indicate poor binding, but this is not the case for nitrophenyl ribosides with the OPRTs. Inhibition assays indicated that p-nitrophenyl β-D-ribose 5′-phosphate was a competitive inhibitor against OMP for both PfOPRT and HsOPRT with Kᵢ values of 40 and 41 nM, respectively (Table 4). Thus, p-nitrophenyl β-D-ribose 5′-phosphate binds substantially better than OMP to give Kₘ/Kᵢ values of 93 and 38 for PfOPRT and HsOPRT, respectively. The role of the phosphate monoester in inhibitor binding was evaluated with p-nitrophenyl β-D-ribose which also inhibited both PfOPRT and HsOPRT to give Kᵢ values of 188 and 121 nM, respectively (Table 4). It is uncommon for nucleotide-binding enzymes to show high affinity without the 5′-phosphate, and the relatively tight binding of p-nitrophenyl 13-D-ribose to both PfOPRT and HsOPRT is a promising lead for the design of cell-permeable analogues that retain strong binding to parasite and/or human OPRTs.

Comparison of Transition State Structures. The transition state of StOPRT has been reported to be D_{N}^{‡}*A_{N}, different from the late associative features (D_{N}*A_{N}^{‡}) of PfOPRT and HsOPRT transition states.³⁷ The transition state of StOPRT has a partially broken C1′-N1 bond with the distance of 1.85 Å and the nucleophile, O^{PA}, is >3.0 Å away from the anomeric carbon. Therefore, the StOPRT transition state is reached prior to full formation of the oxacarbenium ion. In contrast, PfOPRT and HsOPRT transition states show late associative characteristics (D_{N}*A_{N}^{‡}) with full dissociation of orotate and partial participation of the O^{PA} nucleophile. These OPRT transition states can be distinguished by distinct C1′-N1 and C O^{PA} bond orders. The primary [1′-¹⁴C] KIE of 1.03 and α-secondary [1′-³H]KIE of 1.17 have also been reported for yeast OPRT, similar to those of PfOPRT and HsOPRT and suggest a similar dissociative transition state.⁶⁰

PfOPRT and HsOPRT are N-ribosyltransferases with late associative D_{N}*A_{N}^{‡} transition states, similar to that of human 5′-methylthioadenosine phosphorylase (MTAP).⁵⁴ Another group of N-ribosyltransferases, namely those of S. pneumoniae 5′-methylthioadenosine nucleosidase (SpMTAN), E. coli MTAN (EcMTAN), human purine nucleoside phosphorylase (HsPNP) and PfPNP exhibit fully dissociative S_{N}1 transition states with fully developed ribocations. A third group, including N. meningitides MTAN (NmMTAN) and bovine PNP (BtPNP) have early S_{N}¹, partially dissociative D_{N}^{‡}*A_{N} transition states with significant bond order between anomeric carbon and leaving group.^{41,46,52,53,61} These diverse transition state structures among N-ribosyltransferase enzymes can be exploited to develop target-specific transition state analogue inhibitors.

### CONCLUSIONS

The transition state structures of PfOPRT and HsOPRT were determined using density functional calculations constrained by experimental intrinsic KIEs. They have similar transition state structures despite low sequence identity. PfOPRT and HsOPRT transition state structures are characterized by late dissociative D_{N}*A_{N}^{‡} transition states with fully dissociated orotate, ribooxacarbenium ion character, partially formed nucleophilic bonds and 2′-C-endo ribosyl geometry. The lack of catalytic activity with p-nitrophenyl β-D-ribose 5′-phosphate establishes leaving group activation as major force in the reactions catalyzed by PfOPRT and HsOPRT. Despite its poor substrate activity, p-nitrophenyl β-D-ribose 5′-phosphate binds much tighter than the OMP substrate and retains most of the binding energy in the absence of the 5′-phosphate. The PfOPRT and HsOPRT transition states are distinct from that of StOPRT, which featured an early dissociative transition state with no nucleophilic participation. Among the known transition state structures of N-ribosyltransferases, the transition states of PfOPRT and HsOPRT are similar only to that of human MTAP and are distinct from those of StOPRT, SpMTAN, EcMTAN, NmMTAN, HsPNP, PfPNP and BtPNP. The transition state structures of PfOPRT and HsOPRT provide information for the design of transition state analogue inhibitors against the OPRT isozymes.

TABLE 1
Intrinsic KIEs^{a} of PfOPRT and HsOPRT and computationally matched KIEs^{b}.

| Isotopically labeled | | PfOPRT KIEs | | HsOPRT KIEs | |
|---|---|---|---|---|---|
| OMP | KIEs type | Intrinsic | Calcd. | Intrinsic | Calcd. |
| [1′-³H] + [5′-¹⁴C] | α-secondary | 1.261 ± 0.014 | 1.335 | 1.199 ± 0.015 | 1.330 |
| [1′-¹⁴C] + [4′-³H] | primary | 1.034 ± 0.002 | 1.034 | 1.035 ± 0.003 | 1.035 |
| [2′-³H] + [5′-¹⁴C] | β-secondary | 1.116 ± 0.006 | 1.142 | 1.129 ± 0.009 | 1.140 |
| [1,3-¹⁵N₂, 5′-¹⁴C] + [4′-³H] | primary | 1.028 ± 0.003 | 1.025 | 1.025 ± 0.005 | 1.025 |
| [3-¹⁵N, 5′ ¹⁴C] + [4′-³H] | β-secondary | 0.997 ± 0.003 | 1.004 | 0.993 ± 0.002 | 1.004 |
| [1,3-¹⁵N₂, 1′-¹⁴C] + [4′-³H] | primary | 1.068 ± 0.003 | 1.060 | 1.076 ± 0.005 | 1.061 |
| [4′-³H] + [5′-¹⁴C] | γ-secondary | 0.974 ± 0.003 | 0.972 | 0.962 ± 0.002 | 0.972 |
| [5′-³H₂] + [5′-¹⁴C] | δ-secondary | 1.013 ± 0.012 | 1.019 | 1.019 ± 0.002 | 1.018 |

^{a}Intrinsic KIEs were obtained by correcting for remote label KIEs and for forward commitment factors. Experimental KIEs ^{x}(V/K) and intrinsic KIEs ^{x}k are within experimental errors due to the small commitment factors. Each KIE was measured with at least 5 replicates.
^{b}Computationally matched KIEs were established from the Gaussian 98 transition state structures optimized to the family of intrinsic isotope effects. The calculated KIEs compare the bond frequencies from substrate and optimized transition states using the ISOEFF98 program.

TABLE 2
Effect of orotate protonation and/or tautomerization on N1 and N3 KIEs.

| | Intrinsic or Calculated KIEs | | |
|---|---|---|---|
| | N1 | N3 | N1 & N3 |
| HsOPRT | 1.032 ± 0.007 | 0.993 ± 0.002 | 1.025 ± 0.005 |
| PfOPRT | 1.031 ± 0.006 | 0.997 ± 0.003 | 1.028 ± 0.003 |
| Orotate^{a} | 1.021 | 1.004 | 1.025 |
| O2 protonation^{b} | 1.014 | 0.999 | 1.013 |
| O4 protonation^{b} | 1.019 | 1.001 | 1.020 |
| O7 protonation^{b} | 1.019 | 1.003 | 1.022 |
| O—2H tautomer^{a} | 1.018 | 1.014 | 1.032 |
| O—4H tautomer^{a} | 1.022 | 1.014 | 1.037 |
| O—2H tautomer & O7 | 1.017 | 1.013 | 1.030 |
| protonation^{b} | | | |
| O—4H tautomer & O7 | 1.022 | 1.013 | 1.035 |
| protonation^{b} | | | |
| O—2H tautomer & O4 | 1.016 | 1.008 | 1.023 |
| protonation^{b} | | | |
| O—2H tautomer & O4 | 1.014 | 1.006 | 1.020 |
| protonation & O7 | | | |
| protonation^{c} | | | |

^{a}Dianionic.
^{b}Monoanionic.
^{c}Neutral.

TABLE 3
Key geometric changes from the reactants (OMP and PA)(GS) to the
transition states (TS) of PfOPRT and HsOPRT.

| | | PfOPRT | HsOPRT |
|---|---|---|---|
| | bond length (Å) | bond length (Å) | bond length (Å) |
| bond type | (GS) | (TS) | (TS) |
| C1′—N1 | 1.481 | ≧2.600 | ≧2.600 |
| C1′—O^{PA} | >3.0 | 2.150 | 2.140 |
| C1′—O4′ | 1.407 | 1.278 | 1.279 |
| C1′—C2′ | 1.538 | 1.517 | 1.518 |
| C1′—H1′ | 1.092 | 1.082 | 1.082 |
| C2′—H2′ | 1.095 | 1.107 | 1.107 |
| C4′—H4′ | 1.096 | 1.091 | 1.091 |
| C4′—O4′ | 1.456 | 1.530 | 1.530 |
| N1—C2 | 1.401 | 1.358 | 1.358 |
| N1—C6 | 1.393 | 1.355 | 1.355 |
| C2—O2 | 1.221 | 1.252 | 1.252 |
| C2—N3 | 1.389 | 1.416 | 1.416 |
| N3—H3 | 1.011 | 1.011 | 1.011 |
| C4—O4 | 1.232 | 1.257 | 1.257 |

TABLE 4
Kinetic parameters for PfOPRT and HsOPRT with OMP and p-nitrophenyl β-D-ribose
5′-phosphate^{a}.

| | | | | | p-Nitrophenyl | P- |
|---|---|---|---|---|---|---|
| | | | | | β-D-ribose | Nitrophenyl |
| | | OMP | | | 5′-phosphate | β-D-ribose |
| | | k_{cat} | Kₘ | k_{cat}/Kₘ | Kᵢ^{b} | |
| enzyme | substrate | (s⁻¹) | (μM) | (M⁻¹ s⁻¹) | (nM) | |
| PfOPRT | PA | 0.014 ± 0.002 | 2.90 ± 1.09 | 4.8 × 10³ | — | — |
| | PPᵢ | 1.96 ± 0.29 | 3.73 ± 1.36 | 5.3 × 10⁵ | 40 ± 21 | 188 ± 40 |
| HsOPRT | PA | 0.017 ± 0.002 | 2.14 ± 0.61 | 7.9 × 10³ | — | — |
| | PPᵢ | 0.10 ± 0.01 | 1.55 ± 0.61 | 6.5 × 10⁴ | 41 ± 19 | 121 ± 30 |

^{a}Assays were performed at 25° C. by adding purified PfOPRT or HsOPRT into mixtures containing 50 mM Tris-HCl (pH = 8.0), 5 mM MgCl₂, 20 mM PA or 1 mM PPᵢ and varied concentrations of p-nitrophenyl β-D-ribose 5′-phosphate, p-nitrophenyl β-D-ribose, OMP and PA or PPᵢ.
^{b}The Kᵢ value is for the inhibitors with respect to competitive inhibition for OMP.

### REFERENCES

(1) Guerin, P. J.; Olliaro, P.; Nosten, F.; Druilhe, P.; Laxminarayan, R.; Binka, F.; Kilama, W. L.; Ford, N.; White, N. J. Lancet. Infect. Dis. 2002, 2, 564-573.
(2) White, N. J. J. Clin. Invest. 2004, 113, 1084-1092.
(3) ter Kuile, F. O.; van Eijk, A. M.; Filler, S. J. JAMA 2007, 297, 2603-2616.
(4) Jones, M. E. Annu. Rev. Biochem. 1980, 49, 253-279.
(5) Baldwin, J.; Michnoff, C. H.; Malmquist, N. A.; White, J.; Roth, M. G.; Rathod, P. K.; Phillips, M. A. J. Biol. Chem. 2005, 280, 21847-21853.
(6) Krungkrai, S. R.; DelFraino, B. J.; Smiley, J. A.; Prapunwattana, P.; Mitamura, T.; Horii, T.; Krungkrai, J. Biochemistry 2005, 44, 1643-1652.
(7) Krungkrai, S. R.; Aoki, S.; Palacpac, N. M.; Sato, D.; Mitamura, T.; Krungkrai, J.; Horii, T. Mol. Biochem. Parasitol. 2004, 134, 245-255.
(8) Krungkrai, J. Biochim. Biophys. Acta 1995, 1243, 351-360.
(9) Seymour, K. K.; Lyons, S. D.; Phillips, L.; Rieckmann, K. H.; Christopherson, R. I. Biochemistry 1994, 33, 5268-5274.
(10) Krungkrai, J; Krungkrai, S R; Phakanont, K. Biochem. Pharmacol. 1992, 43:1295-1301.
(11) Gero, A. M.; O'Sullivan, W. J. Blood Cells 1990, 16, 467-484.
(12) Schramm, V.; Grubmeyer, C. Prog. Nucleic Acid Res. Mol. Biol. 2004, 78, 261-304.
(13) Henriksen, A; Aghajari, N; Jensen, K F; Gajhede, M. Biochemistry 1996, 35, 3803-9.
(14) Scapin, G.; Ozturk, D. H.; Grubmeyer, C.; Sacchettini, J. C. Biochemistry 1995, 34, 10744-10754.
(15) Grubmeyer, C.; Segura, E.; Dorfman, R. J. Biol. Chem. 1993, 268, 20299-202304.
(16) Suchi, M.; Mizuno, H.; Kawai, Y.; Tsuboi, T.; Sumi, S.; Okajima, K.; Hodgson, M. E.; Ogawa, H.; Wada, Y. Am. J. Hum. Genet. 1997, 60, 525-539.
(17) Yablonski, M. J.; Pasek, D. A.; Han, B. D.; Jones, M. E.; Traut, T. W. J. Biol. Chem. 1996, 271, 10704-10708.
(18) Traut, T. W.; Jones, M. E. Prog. Nucleic Acid Res. Mol. Biol. 1996, 53, 1-78.
(19) Jacquet, M.; Guilbaud, R.; Garreau, H. Mol. Gen. Genet. 1988, 211, 441-445.
(20) Wittmann, J. G.; Heinrich, D.; Gasow, K.; Frey, A.; Diederichsen, U.; Rudolph, M. G. Structure 2008, 16, 82-92.
(21) Quemeneur, L.; Beloeil, L.; Michallet, M. C.; Angelov, G.; Tomkowiak, M.; Revillard, J. P.; Marvel, J. J. Immunol. 2004, 173, 4945-4952.
(22) Chu, E.; Callender, M. A.; Farrell, M. P.; Schmitz, J. C. Cancer Chemother. Pharmacol. 2003, 52 Suppl 1, 80-89.
(23) Christopherson, R. I.; Lyons, S. D.; Wilson, P. K. Acc. Chem. Res. 2002, 35, 961-971.
(24) Allison, A. C. Immunopharmacology 2000, 47, 63-83.
(25) Herrmann, M. L.; Schleyerbach, R.; Kirschbaum, B. J. Immunopharmacology 2000, 47, 273-289.
(26) Weber, G. Biochemistry (Mosc). 2001, 66, 1164-73.
(27) Wolfenden, R.; Snider, M. J. Acc. Chem. Res. 2001, 34, 938-945.
(28) Schramm, V. L. J. Biol. Chem. 2007, 282, 28297-28300.
(29) Schramm, V. L. Curr. Opin. Struct. Biol. 2005, 15, 604-613.
(30) Schramm, V. L. Arch. Biochem. Biophys. 2005, 433, 13-26.
(31) Schramm, V. L. Nucleic Acids Res. Suppl. 2003, 107-108.
(32) Schramm, V. L. Meth. Enzymol. 1999, 308, 301-355.
(33) Schramm, V. L. Acc. Chem. Res. 2003, 36, 588-596.
(34) Cleland, W. W. Arch. Biochem. Biophys. 2005, 433, 2-12.
(35) Mazzella, L. J.; Parkin, D. W.; Tyler, P. C.; Furneaux, R. H.; Schramm, V. L. J. Am. Chem. Soc. 1996, 118, 2111-2112.
(36) Knecht, W.; Petersen, G. E.; Sandrini, M. P.; Sondergaard, L.; Munch-Petersen, B.; Piskur, J. Nucleic Acids Res. 2003, 31, 1665-72.
(37) Tao, W.; Grubmeyer, C.; Blanchard, J. S. Biochemistry 1996, 35, 14-21.
(38) Rising, K. A.; Schramm, V. L. J. Am. Chem. Soc. 1994, 116, 6531-6536.
(39) Luo, M.; Schramm, V. L. J. Am. Chem. Soc. 2008.
(40) Luo, M; Singh, V; Taylor, E A; Schramm, V L. J. Am. Chem. Soc. 2007, 129, 8008-17.
(41) Singh, V.; Lee, J. E.; Nunez, S.; Howell, P. L.; Schramm, V. L. Biochemistry 2005, 44, 11647-11659.
(42) Parkin, D. W.; Leung, H. B.; Schramm, V. L. J. Biol. Chem. 1984, 259, 9411-9417.
(43) Fox, S. W.; Johnson, J. E. Science 1956, 124, 923-5.
(44) Takeyama, S.; Noguchi, T.; Miura, Y.; Ishidate, M. Pharm. Bull. 1956, 4, 492-494.
(45) Luo, M.; Li, L.; Schramm, V. L. Biochemistry 2008, 47, 2565-76.
(46) Lewandowicz, A.; Schramm, V. L. Biochemistry 2004, 43, 1458-1468.
(47) Frisch, M. J. et al.; Gaussian, Inc.: Pittsburgh Pa., 2002.
(48) Anisimov, V.; Paneth, P. J. Math. Chem. 1999, 26, 75-86.
(49) Flükiger, P.; Lüthi, H. P.; Portmann, S.; Weber, J.; Swiss Center for Scientific Computing, M., Switzerland, 2000-2002.
(50) Portmann, S.; Lüthi, H. P. CHIMIA 2000, 54, 766-770.
(51) Northrop, D. B. Biochemistry 1975, 14, 2644-2651.
(52) Singh, V.; Schramm, V. L. J. Am. Chem. Soc. 2007, 129, 2783-2795.
(53) Singh, V.; Luo, M.; Brown, R. L.; Norris, G. E.; Schramm, V. L. J. Am. Chem. Soc. 2007, 129, 13831-13833.
(54) Singh, V.; Schramm, V. L. J. Am. Chem. Soc. 2006, 128, 14691-14696.
(55) Scapin, G.; Grubmeyer, C.; Sacchettini, J. C. Biochemistry 1994, 33, 1287-1294.
(56) Gonzalez-Segura, L.; Witte, J. F.; McClard, R. W.; Hurley, T. D. Biochemistry 2007, 46, 14075-14086.
(57) Berti, P. J.; Tanaka, K. S. E. Adv. Phys. Org. Chem. 2002, 37, 239-314.
(58) Pham, T. V.; Fang, Y. R.; Westaway, K. C. J. Am. Chem. Soc. 1997, 119, 3670-3676.
(59) McCann, J. A.; Berti, P. J. J. Am. Chem. Soc. 2007, 129, 7055-7064.
(60) Goitein, R. K.; Chelsky, D.; Parsons, S. M. J. Biol. Chem. 1978, 253, 2963-2971.
(61) Kline, P. C.; Schramm, V. L. Biochemistry 1993, 32, 13212-13219.
(62) Bagdassarian, C. K., Schramm, V. L., and Schwartz, S. D. J. Am. Chem. Soc 1996 118, 8825-8836.

1. A method for designing a transition state inhibitor of orotate phosphoribosyltransferase (OPRT) comprising designing a compound that resembles the charge and geometry of the OPRT transition state.

2. The method of claim 1, wherein the OPRT has intrinsic kinetic isotope effects for [1′-¹⁴C], [1, 3-¹⁵N₂], [3-¹⁵N], [1′-³H], [2′-³H], [4′-³H] and [5′-³H₂] of 1.034, 1.028, 0.997, 1.261, 1.116, 0.974 and 1.013, respectively, or wherein the OPRT has intrinsic kinetic isotope effects for [1′-¹⁴C], [1, 3-¹⁵N₂], [3-¹⁵N], [1′-³H], [2′-³H], [4′-³H] and [5′-³H₂] of 1.035, 1.025, 0.993, 1.199, 1.129, 0.962 and 1.019, respectively.

3. The method of claim 1, wherein the OPRT is a Plasmodium OPRT or a human OPRT.

4. The method of claim 1, wherein the OPRT transition state is characterized by one or more of a late dissociative D_{N}*A_{N}^{‡} transition state, a fully dissociated orotate, a ribooxacarbenium ion character, partially formed nucleophilic bonds, 2′-C-endo ribosyl geometry, and a C1′-O^{PA} distance of about 2.1 Å.

5. The method of claim 1, wherein the OPRT transition state is characterized by two or more of a late dissociative D_{N}*A_{N}^{‡} transition state, a fully dissociated orotate, a ribooxacarbenium ion character, partially formed nucleophilic bonds, 2′-C-endo ribosyl geometry, and a C1′-O^{PA} distance of about 2.1 Å.

6. The method of claim 1, wherein the OPRT transition state is characterized by a late dissociative D_{N}*A_{N}^{‡} transition state, a fully dissociated orotate, a ribooxacarbenium ion character, partially formed nucleophilic bonds, 2′-C-endo ribosyl geometry, and a C1′-O^{PA} distance of about 2.1 Å.

7. The method of claim 1, which comprises designing a chemically stable compound that resembles (a) the molecular electrostatic potential at the van der Walls surface computed from the molecular wave function of the transition state of OPRT and (b) the geometric atomic volume of the OPRT transition state.

8. The method of claim 1, wherein the compound comprises a moiety resembling the molecular electrostatic potential surface of the ribosyl group at the transition state.

9. The method of claim 1, which further comprises analyzing the compound for its ability to inhibit OPRT.

10. A method of inhibiting orotate phosphoribosyltransferase (OPRT) comprising designing a transition state inhibitor of OPRT by the method of claim 1 and then contacting the OPRT with the inhibitor.

11. The method of claim 10, wherein the OPRT is in a parasite or in a human cell.

12. The method of claim 11, wherein the human cell is a cancer cell.

13. The method of claim 11, wherein the human cell is in a subject suffering from an autoimmune disease.

14. The method of claim 11, wherein the parasite is a protozoan parasite of the genus Plasmodium.

15. The method of claim 14, wherein the parasite is Plasmodium falciparum, Plasmodium vivax, Plasmodium ovale or Plasmodium malariae.
